(19) United States
(12) Patent Application Publication
(10) Pub. No.: US 2016/0175221 A1
(43) Pub. Date: Jun. 23, 2016

(54) COMPOSITION FOR PREVENTING SKIN AGING CONTAINING ALOESIN OR DERIVATIVE THEREOF

(71) Applicants: UNIVERA INC., Seoul (KR); GACHON UNIVERSITY OF INDUSTRY-ACADEMIC COOPERATION FOUNDATION, Gyeonggi-do (KR)

(72) Inventors: Seon-Gil DO, Seoul (KR); Jin-Wan KIM, Namyangju-si, Gyeonggi-do (KR); Yoo-Jeong JIN, Seongnam-si, Gyeonggi-do (KR); Sun-Yeou KIM, Seoul (KR); Ji-Hye MOON, Incheon (KR); Choong-Hwan LEE, Seoul (KR); Sa-Rah LEE, Seoul (KR)

(21) Appl. No.: 14/909,137

(22) PCT Filed: Jul. 11, 2014
(86) PCT No.: PCT/KR2014/006246

§ 371 Date: Jan. 31, 2016

(30) Foreign Application Priority Data
Jul. 31, 2013 (KR) 10-2013-0090595

(51) Int. Cl. A61K 8/60; A61K 8/97; A61Q 17/04

(52) U.S. Cl. CPC A61K 8/602; A61Q 17/04; A61K 8/97

(57) ABSTRACT

The present invention relates to a composition for preventing skin aging containing aloesin or a derivative thereof. The aloesin or the derivative thereof, according to the present invention reduces cytotoxicity and inhibits a MMP-1 level and increases PIP levels and procollagen type 1, in normal human dermal fibroblasts (NHDF) after ultraviolet irradiation, and thus is particularly effective for preventing or treating skin photoaging induced by ultraviolet irradiation.

### TECHNICAL FIELD

The present invention relates to a composition for preventing skin aging containing aloesin or a derivative thereof.

### BACKGROUND ART

Ultraviolet (UV) radiation is composed of three wavelengths: ultraviolet A (UVA; 320-400 nm), ultraviolet B (UVB; 290-320 nm) and ultraviolet C (UVC; 100-280 nm). It is considered that excessive or repeated exposure to ultraviolet radiation, particularly to ultraviolet B, is a major cause of sunburn or skin damage. The most remarkable changes such as destruction of matrix components including collagen type 1, elastin, proteoglycan and fibronectin can be found in the dermis of photo-aged skin (Fisher et al., 2002; Fisher et al., 2009). Degradation of collagen induced by matrix metalloproteinases (MMPs) and other signs of skin photoaging do not cause the skin completely to return to its original state even by means of collagen synthesis (Quan et al., 2009). Degradation of structural extracellular matrix (ECM) increases expression of MMPs known as interstitial collagenases. Cherng et al., have reported that induction of interleukin-6 (IL-6) by UVB irradiation especially affects the protein levels of matrix metalloproteinase-1 (MMP-1) and matrix metalloproteinase-3 (MMP-3) as target collagen type I and III. It is well known that transforming growth factor-β1 (TGF-β1) is a major factor for regulating synthesis of procollagen type 1 in dermal fibroblasts (Quan et al., 2002).

Among 500 species of aloe, only a few species including aloe vera have been cultivated in hot climates such as southern Texas in USA; Mexico; India; and Africa (Moghaddasi and Verm, 2011). Aloe vera is also grown on Cheju-island in Korea.

Aloe barbadensis M. (Aloe vera) is a plant belonging to family Asphodelaceae and has been used as a raw material for functional foods, cosmetics, and herbal medicines (Wynn et al., 2005; Djuv and Nilsen, 2012; Shimpo et al., 2002). Aloe vera is known to include aloin, aloe-emodin and aloesin as a polyphenolic structure presumed to have biological efficiencies including antioxidant effects (Wamer et al., 2003; Bawankar et al., 2012). Particularly, aloesin is a competitive tyrosinase inhibitor and has been observed to exhibit free radical scavenging activity (Jones et al., 2002; Yagi et al., 2002). Many researchers have focused on dermatological efficacies of aloe vera including moisturizing effects, anti-inflammatory effect, anti-scabies, anti-psoriatic activities and wound treatment (Dal'sBelo et al., 2006; Byeon et al., 1998; Oyelami et al., 2009; Dhanabal et al., 2012; John et al., 1995).

However, there has been no research reporting efficacy of aloe vera, particularly, aloesin or a derivative thereof protecting the skin from photoaging induced by ultraviolet radiation.

### DISCLOSURE

### Technical Problem

Embodiments of the present invention provide a composition for preventing skin aging containing aloesin or a derivative thereof.

However, it should be understood that technical problems to be achieved by the present invention are not restricted thereto, and other problems not mentioned will be apparent to those skilled in the art from the following disclosure.

### Technical Solution

The present invention provides a composition for preventing skin aging containing a compound represented by the following Formula 1.

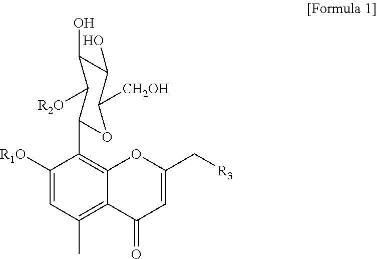

wherein R₁ is H or CH₃, R₂ is H or ρ-coumaroyl, and R₃ is COCH₃ or CH(OH)CH₃.

### Advantageous Effects

The present invention relates to a composition for preventing skin aging containing aloesin or a derivative thereof. Aloesin or the derivative thereof according to the present invention is effective for preventing or treating skin photoaging induced by ultraviolet irradiation since aloesin or the derivative thereof can reduce cytotoxicity in normal human dermal fibroblasts (NHDFs), inhibit MMP-1 synthesis, and increase type I procollagen peptide (PIP) levels and procollagen type I levels after ultraviolet irradiation.

### DESCRIPTION OF DRAWINGS

FIG. 1a shows cytotoxicity in non-treated human dermal fibroblasts prior to UVB irradiation; and cytotoxicity in aloesin, aloeresin A and isoaloeresin D treated human dermal fibroblasts prior to UVB irradiation, and FIG. 1b shows cytotoxicity in non-treated human dermal fibroblasts prior to UVB irradiation; cytotoxicity in non-treated human dermal fibroblasts after UVB irradiation; and cytotoxicity in aloesin, aloeresin A and isoaloeresin D treated human dermal fibroblasts after UVB irradiation.

FIG. 2 shows MMP-1 levels in non-treated human dermal fibroblasts prior to UVB irradiation; MMP-1 levels in non-treated human dermal fibroblasts after UVB irradiation; and MMP-1 levels in aloesin, aloeresin A and isoaloeresin D treated human dermal fibroblasts after UVB irradiation.

FIG. 3a shows PIP levels in non-treated human dermal fibroblasts prior to UVB irradiation; and PIP levels in aloesin, aloeresin A and isoaloeresin D treated human dermal fibroblasts prior to UVB irradiation, and FIG. 3b shows PIP levels in non-treated human dermal fibroblasts prior to UVB irradiation; PIP levels in non-treated human dermal fibroblasts after UVB irradiation; and PIP levels in aloesin, aloeresin A and isoaloeresin D treated human dermal fibroblasts after UVB irradiation.

FIG. 4 shows mRNA levels of MMP-1 and procollagen type I in non-treated human dermal fibroblasts prior to UVB irradiation; mRNA levels of MMP-1 and procollagen type I in non-treated human dermal fibroblasts after UVB irradiation; and mRNA levels of MMP-1 and procollagen type I in aloesin treated human dermal fibroblasts after UVB irradiation.

### BEST MODE

The present inventors have made investigation for skin aging prevention efficacy of Aloe vera using aloesin or a derivative thereof isolated from Aloe vera extracts in the adjustment of skin damage caused by ultraviolet radiation.

The present invention provides a composition for preventing skin aging containing a compound represented by the following Formula 1.

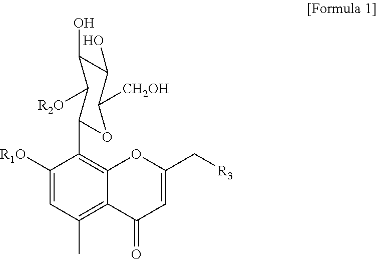

wherein R₁ is H or CH₃, R₂ is H or ρ-coumaroyl, and R₃ is COCH₃ or CH(OH)CH₃.

The compound represented by Formula 1 can be isolated from Aloe vera extracts. The extracts may be extracted using a solvent selected from the group consisting of water, an anhydrous or hydrated C₁ to C₄ lower alcohol, a C₁ to C₄ lower ketone, a C₁ to C₄ lower ester, chloroform, and mixtures thereof, without being limited thereto.

The compound represented by Formula 1 may be aloesin, aloeresin A, isoaloeresin D, or 7-O-methylaloeresin A.

Specifically, in Formula 1, aloesin is a compound wherein R₁ is H, R₂ is H, and R₃ is COCH₃.

Aloeresin A, isoaloeresin D and 7-O-methylaloeresin A are derivatives of aloesin. In Formula 1, aloeresin A is a compound wherein R₁ is H, R₂ is ρ-coumaroyl, and R₃ is COCH₃; isoaloeresin D is a compound wherein R₁ is CH₃, R₂ is ρ-coumaroyl, and R₃ is CH(OH)CH₃; and 7-O-methylaloeresin A is a compound wherein R₁ is CH₃, R₂ is ρ-coumaroyl, and R₃ is COCH₃.

Skin aging includes natural aging or photoaging, wherein natural aging is induced by internal changes while photoaging is induced by external changes.

Skin aging may be skin photoaging induced by ultraviolet irradiation.

The ultraviolet radiation may be radiation by ultraviolet B (UVB) having a wavelength of 290 nm to 320 nm.

The composition for preventing skin aging according to the present invention may be provided as a cosmetic composition or a pharmaceutical composition.

The compound represented by Formula 1 may be present in a dosage amount of 0.01 μg/ml to 25 μg/ml.

As such, when the composition for preventing skin aging according to the present invention is provided as either a cosmetic composition or a pharmaceutical composition, the composition may further include suitable carriers, excipients, diluents, and the like, which are conventionally used in the preparation of cosmetic compositions or pharmaceutical compositions.

Namely, the cosmetic composition or pharmaceutical composition may be formulated in various forms including external preparations such as ointments, gels, creams, patches, sprays, and the like in accordance with typical methods. Each formulation may contain various appropriate base materials and additives which are required in formulating such a preparation. The sorts and amounts of these components can be easily selected by the inventors.

Accordingly, the present invention relates to a composition for preventing skin aging containing aloesin or a derivative thereof. The aloesin or the derivative thereof according to the present invention is effective in the prevention or treatment of skin photoaging induced by ultraviolet irradiation since aloesin or the derivative thereof can reduce cytotoxicity in normal human dermal fibroblasts (NHDF), inhibit MMP-1 levels, and increase type I procollagen peptide (PIP) levels and procollagen type I levels after ultraviolet irradiation.

Hereinafter, the present invention will be described in more detail with reference to some examples. It should be understood that these examples are provided for illustration only and are not to be construed in any way as limiting the present invention.

### EXAMPLES

### Isolation of Aloesin and Aloesin Derivatives from Aloe Vera Extracts

Aloe vera was extracted with 400 ml of methanol for 12 hours by employing a twist shaker (manufactured by BioFree, Korea) to yield a dried sample. After extraction, the extract was subjected to centrifugation at 3000 rpm at 4° C. for 10 minutes. 1 ml of the supernatant liquid was completely dried in a high-speed vacuum concentrator (manufactured by Biotron, Korea) for 12 hours. In order to perform Liquid Chromatography-Mass Spectrometry (LC-MS) analysis, the dried sample was dissolved in 1 ml of methanol, and then was filtered by passing through a 0.2 μm of polytetrafluoroethylene filter.

Ultra Performance Liquid Chromatography (UPLC) was performed on a Waters ACQUITY UPLC System (manufactured by Waters Co., Ltd., Mildford, Mass.) equipped with a binary solvent manager, an auto-sampler and a UV detector. Chromatographic separation was performed on a Waters ACQUITY HLPC BEH C18 column (100×2.1 mm i.d., 1.7 μm particle size). Elution was performed using acetonitrile (ACN)/water containing 0.1% formic acid. Particles were linearly increased from 0% to 90% within 12 minutes, and then were decreased to 0% after 3 minutes. Total run time including re-equilibration of the column under starting conditions was 17 minutes. The injected amount was 5 μl and the flow rate was 0.3 ml/min. In order to perform Mass Spectrometry (MS), a Waters G-TOF Premier (manufactured by Micromass MS Technologies, Manchester, England) was operated in a wide pass quadrupole mode equipped with TOF data collected from a range of m/z 100-1500 in both negative and positive ion modes. The de-solvation gas (nitrogen) was set to 600 l/h at a temperature of 200° C.; the cone gas (nitrogen) was set to 50 l/h; and the feed temperature was set to 100° C. The capillary and cone voltages were set to 3.0 kV and 40V, respectively. Data were collected in the centroid mode with 0.2 second scan accumulation time. Compounds (aloesin, aloeresin A, isoaloeresin D, 7-O-methylaloeresin A) were compared in view of mass spectrum and retention time and clearly identified using standard compounds.

Cell Culture

Normal human dermal fibroblasts (NHDFs) were harvested from a young healthy male donor (MCTT Core, Inc., Seoul, Korea) by skin biopsy. The cells were plated on 100-mm tissue culture plates, and cultured in Dulbeco's modified Eagle's medium (DMEM) supplemented with 10% heat-inactivated fetal bovine serum (FBS) and 1% penicillin-streptomycin at 37° C. in a humid environment containing 5% CO2. For all experiments only those cells between passages 6 and 10 were used.

UVB Irradiation and Treatment of Cells with Aloesin or Aloesin Derivative

Normal human dermal fibroblasts were seeded into 40-mm tissue culture plates (1.2×10⁵ cells). When normal human dermal fibroblasts reached 80% confluence, they were washed with phosphate buffered saline (PBS) twice. All irradiation was conducted under a thin layer of PBS with the plates closed. UVB irradiation was supplied by a closely spaced array of five sunlamps (Sankyo Denki Co.), which delivered uniform irradiation at a distance of 7.5 cm. Irradiation (144 mJ/cm²) was measured with a UVB photometer (IL1700 photometer, International Light). After irradiation, a fresh serum-free medium (1,980 μl) and a test sample (20 μl) were added to each well. Then, normal human dermal fibroblasts were washed with warm phosphate buffered saline (PBS) three times. Normal human dermal fibroblasts of control group were stored under the same culture conditions without UVB exposure. The production of MMP-1 and PIP was evaluated in a supernatant liquid harvested 72 hours after UVB irradiation. For reverse transcription polymerase chain reaction (RT-PCR) analysis, normal human dermal fibroblasts were harvested 24 hours after UVB irradiation.

Statistical Analysis

All experiments were conducted in triplicate. The data were expressed as mean±SD values. Statistical comparison between different treatments was performed using a one-way analysis of variance (ANOVA) by Duncan's test. For statistical analysis, Student's T-test to compare individual treatments to the controls was conducted. Statistical significance was set to P<0.05.

### Example 1

### Identification of Cytotoxicity of Aloesin Treated Cells

3-(4,5-dimethylthiazol-2-yl)-2,5-diphenyltetrazolium bromide (MTT) colorimetric assay is a commonly used method to determine cell viability. MTT turns into formazan dyes capable of exhibiting purple color. 72 hours after incubation, volume of the medium was reduced to 1 ml. 100 μl of MTT (1 mg/ml) was added to each well. Then, cells were incubated at 37° C. for 2 hours in the presence of 5% CO₂ and 95% O₂. The substrate-containing medium was removed and 1 ml of dimethyl sulfoxide (DMSO) was added to each well to dissolve the formazan crystals. The plates were then agitated at room temperature on an orbital shaker for 30 minutes. Absorbance of 100 μl aliquots was measured using a microplate reader (E09090, Molecular Devices, San Francisco, Calif., USA) at a wavelength of 570 nm.

In order to identify cytotoxicity of aloesin treated cells (72 hours after treatment), MTT assay was performed in UVB irradiated normal human dermal fibroblasts.

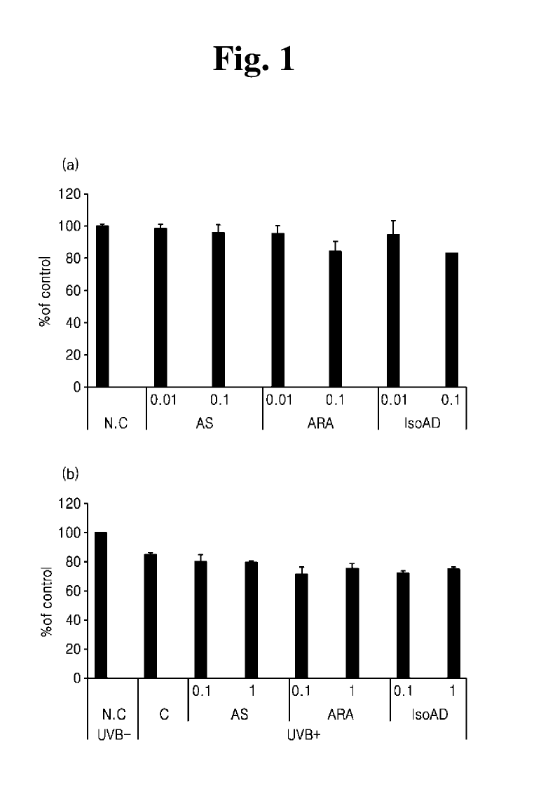

FIG. 1a shows cytotoxicity in non-treated (N.C.) human dermal fibroblasts prior to UVB irradiation; and cytotoxicity in aloesin (AS), aloeresin A (ARA) and isoaloeresin D (IsoAD) (0.01 μg/ml and 0.1 μg/ml) treated human dermal fibroblasts prior to UVB irradiation, and FIG. 1b shows cytotoxicity in non-treated (N.C) human dermal fibroblasts prior to UVB irradiation; cytotoxicity in non-treated (C) human dermal fibroblasts after UVB irradiation; and cytotoxicity in aloesin (AS), aloeresin A (ARA) and isoaloeresin D (IsoAD) (0.1 μg/ml and 1 μg/ml) treated human dermal fibroblasts after UVB irradiation.

As depicted in FIG. 1, it was confirmed that aloesin (AS), aloeresin A (ARA) and isoaloeresin D (IsoAD) treated human dermal fibroblasts exhibited decreased cytotoxicity before and after UVB irradiation.

### Example 2

### Identification of MMP-1 and PIP Levels of Aloesin Treated Cells

The concentrations of MMP-1 and PIP in the medium were determined using commercially available enzyme-linked immunosorbent assay (ELISA) kits (Human Total MMP-1 Kit, R&D Systems, Inc., Minneapolis, Minn., USA and procollagen type I C-peptide enzyme immunoassay (EIA) kit, Takara Inc., Shiga, Japan) according to the manufacturer's instructions. Each sample was analyzed in triplicate.

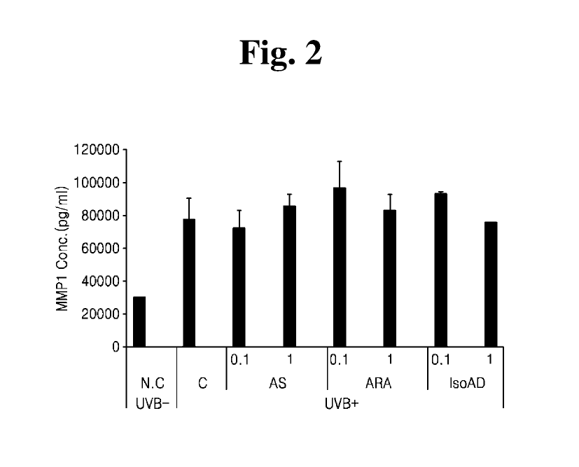

FIG. 2 shows MMP-1 levels in non-treated (N.C) human dermal fibroblasts prior to UVB irradiation; MMP-1 levels in non-treated (C) human dermal fibroblasts after UVB irradiation; and MMP-1 levels in aloesin (AS), aloeresin A (ARA) and isoaloeresin D (IsoAD) treated human dermal fibroblasts after UVB irradiation.

As depicted in FIG. 2, it was confirmed that MMP-1 levels were greatly increased within 72 hours after UVB irradiation. Specifically, it was also confirmed that aloesin (AS) treated (0.1 μg/ml) or isoaloeresin D (IsoAD) treated (1 μg/ml) normal human dermal fibroblasts exhibit significantly decreased MMP-1 levels, which was increased by UVB irradiation.

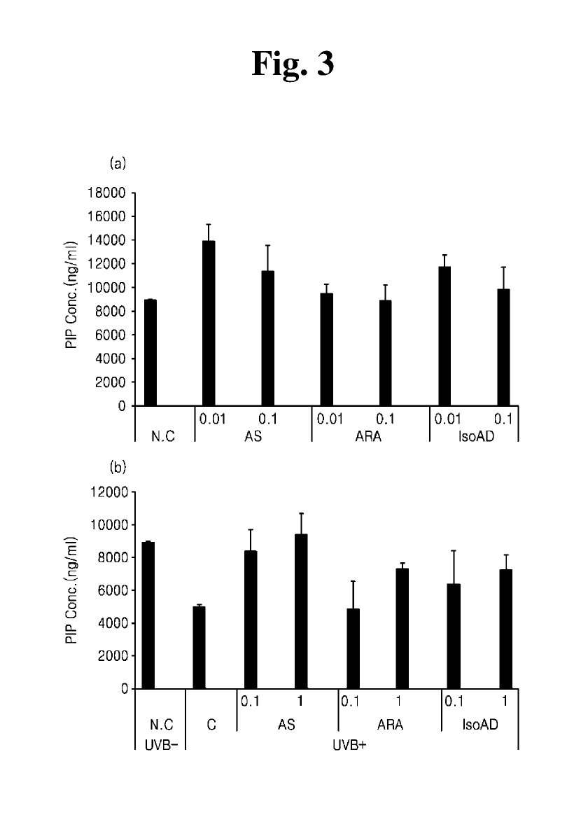

FIG. 3a shows PIP levels in non-treated (N.C) human dermal fibroblasts prior to UVB irradiation; and PIP levels in aloesin (AS), aloeresin A (ARA) and isoaloeresin D (IsoAD) treated (0.01 μg/ml and 0.1 μg/ml) normal human dermal fibroblasts prior to UVB irradiation.

As depicted in FIG. 3a, aloesin (AS), aloeresin A (ARA) and isoaloeresin D (IsoAD) treated (0.01 μg/ml and 0.1 μg/ml) normal human dermal fibroblasts exhibited significantly increased PIP levels. Namely, it is apparent that even before UV irradiation, treatment of cells with aloesin or aloesin derivatives was effective in inhibiting skin aging.

Further, FIG. 3b shows PIP levels in non-treated (N.C.) human dermal fibroblasts prior to UVB irradiation; PIP levels in non-treated (C) human dermal fibroblasts after UVB irradiation; and PIP levels in aloesin (AS), aloeresin A (ARA) and isoaloeresin D (IsoAD) treated (0.1 μg/ml and 1 μg/ml) human dermal fibroblasts after UVB irradiation.

As depicted in FIG. 3b, it was confirmed that PIP levels were greatly decreased within 72 hours after UVB irradiation. In addition, it was confirmed that aloesin (AS), aloeresin A (ARA) and isoaloeresin D (IsoAD) treated (0.01 μg/ml and 0.1 μg/ml) normal human dermal fibroblasts significantly increased PIP levels which were decreased by UVB irradiation. Namely, it is apparent that after UV irradiation and treatment of cells with aloesin or aloesin derivatives was effective to inhibit skin aging.

### Example 3

### Identification of mRNA Levels of MMP-1 and Procollagen Type I in Aloesin Treated Cells

After UVB irradiation, separation of RNA from aloesin treated normal human dermal fibroblasts (NHDFs) was performed using TRIZOL reagent (Invitrogen Life Technologies, Carlsbad, Canada) according to the manufacturer's instructions. RNA (5 μg) was subjected to reverse transcription using 200 units of reverse transcriptase and 0.5 μg/μl of oligo-(dT) 15 primer (Bioneer Inc., Korea). The reaction was performed at 42° C. for 60 minutes, and then terminated by heating at 94° C. for five minutes. PCR amplification of cDNA template was performed using PCR premix (Bioneer Inc.) and the following primer pairs: MMP-1, forward 5′-ATT CTA CTG ATA TCG GGG CTT TGA-3′, reverse 5′-ATG TCC TTG GGG TAT CCG TGT AG-3′; procollagen type I, forward 5′-CTC GAG GTG GAC ACC ACC CT-3′, reverse 5′-CAG CTG GAT GGC CAC ATC GG-3′; and glyceraldehydes-3-phosphate dihydrogenase (GAPDH), sense 5′-ACC ACA GTC CAT GCC ATC AC-3′, antisense 5′-CCA CCA CCC TGT TGC TGT AC-3′. PCR was performed in a Veriti Thermal Cycler (Applied Biosystems, Foster city, CA, USA) for 30 cycles. PCR products were separated by electrophoresis under UV light on 2.0% agarose gels stained with ethidium bromide. Glyceraldehydes-3-phosphate dihydrogenase (GAPDH) was used as an internal control. Each experiment was conducted in at least triplicate.

To investigate mRNA levels of aloesin treated cells (24 hours after treatment), the concentration of MMP-1 and procollagen type I was measured in UVB irradiated normal human dermal fibroblasts. To quantify the data, the ratio of MMP-1/GAPDH and procollagen type I/GAPDH in normal human dermal fibroblasts was randomly set to 1.0, based on band signal intensity.

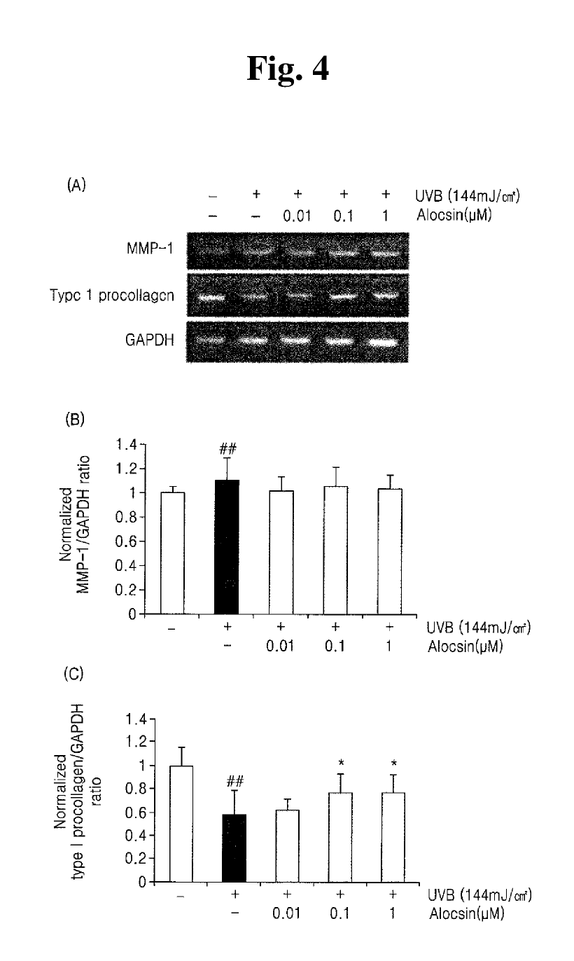

FIG. 4 shows mRNA levels of MMP-1 and procollagen type I in non-treated human dermal fibroblasts prior to UVB irradiation; mRNA levels of MMP-1 and procollagen type I in non-treated human dermal fibroblasts after UVB irradiation; and mRNA levels of MMP-1 and procollagen type 1 in aloesin treated (0.01 μg/ml, 0.1 μg/ml and 1 μg/ml) human dermal fibroblasts after UVB irradiation.

As depicted in FIG. 4, it was confirmed from RT-PCR data that UVB irradiation increased mRNA levels of MMP-1 within 24 hours, whereas decreased mRNA levels of procollagen type I. As depicted in FIG. 4c, it was confirmed that aloesin treated (0.01 μg/ml, 0.1 μg/ml and 1 μg/ml) normal human dermal fibroblasts decreased mRNA expression of MMP-1 which was increased by UVB irradiation and significantly increased mRNA expression of procollagen type I, which was decreased by UVB irradiation.

Although some embodiments have been described herein, it should be understood that these embodiments are given by way of illustration only, and that various modifications, variations, and alterations can be made by those skilled in the art without departing from the spirit and scope of the invention. Therefore, the scope of the invention should be limited only by the accompanying claims and equivalents thereof.

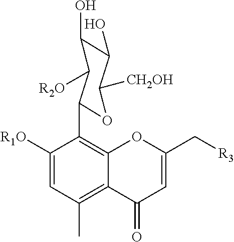

1. A composition for preventing skin aging comprising a compound represented by the following Formula 1. 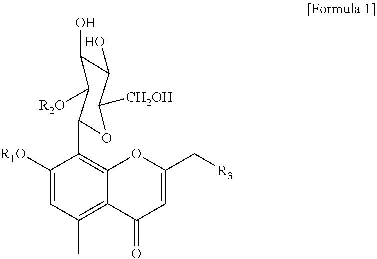
wherein R₁ is H or CH₃, R₂ is H or ρ-coumaroyl, and R₃ is COCH₃ or CH(OH)CH₃.

2. The composition for preventing skin aging according to claim 1, wherein the compound represented by Formula 1 is isolated from aloe vera extract.

3. The composition for preventing skin aging according to claim 2, wherein the extract is extracted using a solvent selected from the group consisting of water, an anhydrous or hydrated C₁ to C₄ lower alcohol, a C₁ to C₄ lower ketone, a C₁ to C₄ lower ester, chloroform and mixtures thereof.

4. The composition for preventing skin aging according to claim 1, wherein the compound represented by Formula 1 is aloesin, aloeresin A, isoaloeresin D, or 7-O-methylaloeresin A.

5. The composition for preventing skin aging according to claim 1, wherein the skin aging is skin photoaging induced by ultraviolet irradiation.

6. The composition for preventing skin aging according to claim 5, wherein the ultraviolet radiation is ultraviolet B (UVB) having a wavelength of 290 nm to 320 nm.

7. The composition for preventing skin aging according to claim 1, wherein the compound represented by Formula 1 is present in a dosage amount of 0.01 μg/ml to 25 μg/ml.
